(19) United States
(12) Patent Application Publication
(10) Pub. No.: US 2012/0328537 A1
(43) Pub. Date: Dec. 27, 2012

(54) Topical wound management formulation

(76) Inventor: Richard A. Cloonan, San Marcos, CA (US)

(21) Appl. No.: 13/167,421

(22) Filed: Jun. 23, 2011

(51) Int. Cl. A61K 8/97; A61K 31/047; A61K 36/534; A61K 36/886; A61P 17/02; A61C 15/04; A61Q 11/00; A61P 17/10; A61P 17/00; A61P 17/06; A61C 17/00; A61K 8/84; A61P 31/00

(52) U.S. Cl. 424/58; 424/49; 514/738; 424/747; 514/729; 424/744; 433/216

(57) ABSTRACT

A topical wound therapeutic treatment provides a gel preparation of very low toxicity and good antimicrobial properties. The gel can be formulated for both intra-oral and extra-oral use. The preparation uses high concentration Xylitol in combination with other agents including a non-Xylitol, sugarless, antimicrobial sweetener, such as DL-Menthol, oil of Peppermint, Sorbitol and Glycerin to achieve rapid wound healing with comfort. The dried gel forms a water soluble occlusive dressing to promote healing.

### FIELD OF THE INVENTION

This invention relates to personal hygiene preparations and more particularly to the treatment of topical wounds.

### BACKGROUND

Superficial topical wounds can occur either intra-orally and extra-orally. Open wounds provide a pathway for the entry of pathogenic microorganisms into the body which can lead to infection and other conditions which prolong healing. The causes of open wounds can range from physical cuts and abrasions to bodily manifestations of disease. Conditions of the skin such as acne, bacterial and fungal infections, diaper rash, sunburn, dandruff, and conditions of the mouth such as tooth decay and thrush can lead to the creation of wounds, and mouth ulcers which can lead to skin infections, canker sores and gingivitis for example. Further, when a person's immune system is weakened, pathogens may colonize and invade susceptible tissues. It is well known that poor oral health can lead to other seemingly unrelated unhealthful conditions in the body such as heart disease as an example.

It is well known that even the healthy mouth is home to scores of potentially harmful pathogenic microorganisms such as strep mutans. Typically, in a healthy individual, the body's own defenses will typically keep populations of such organisms within acceptable limits. However, a mouth having a more acidic pH environment will tend to break down the protective layer of Fibronectin on mouth tissues leading to the growth of strep mutans. It has been reported that at pH 4.0 normal bacterial flora in the mouth can be displaced by pathogenic bacteria leading to pathogen colonization. Schaechter M., Ph.D., et al., Mechanisms of Microbial Disease, 1999, Lippincott Williams & Wilkins, pp 13-15, 32. It is well known that the environment of the mouth becomes more acidic after meals.

It is well-known that during surgery a person can be particularly susceptible to attack by pathogens because certain defensive pathways in the body have been diminished or bypassed. Although surgeons have taken great efforts to reduce the presence of pathogens in the operating room by fastidious washing and sterilization of equipment, the common pathogens present in the patient's mouth such as strep mutans and staph aureus are often overlooked and act as a source for transmission of these pathogens. For example, it has been found that use of an endotracheal tube can lead to a condition known as “aspiration pneumonia” where pathogens from the mouth have been carried into the lungs by the tube.

It has been reported that the rate of respiratory infection among critically ill patients increases when there is an increase in the presence of disease causing bacteria in the mucosal areas of the mouth, gums, and teeth. S. Pear, Ph.D., et al., The Role of Oral Care in the Prevention of Hospital Acquired Pneumonia, The American Association of Critical Care Nurses, October 2007.

Fluoride's ability to remineralize enamel and provide an antimicrobial effect is well-known. However, despite the emphasis on preventive dentistry and the introduction of dentifrices and mouth washes having high levels of Fluoride, mouth pH levels often become acidic enough for common mouth pathogens to colonize.

Further, high levels of fluoride in toothpaste, prescription mouthwashes and prescription gels are not without risks. Naturally occurring Fluoride levels of 10 to 12 parts per million in water found in certain areas of Texas and new Mexico have been blamed for the mottling of tooth enamel and for high incidence of osteosclerosis. The toxicity of Fluoride creates serious risks for the safety of small children and disabled individuals who occasionally swallow tooth paste while brushing teeth. Indeed, containers of most popular brands of toothpaste carry warning legends urging customers to seek professional help or contact a poison control center immediately upon accidental ingestion of more toothpaste than is used for brushing.

Patients with compromised immune systems, such as those suffering from Acquired Immune Deficiency (AIDS) or those undergoing chemotherapeutic treatments are particularly suspectible to mouth ulcers and irritations. Such persons are in great need of a dental preparation that can be used to thoroughly rinse their teeth and mouth, stimulate healing of lesions, and be devoid of any poisoning risks.

Studies have shown that high amounts of Xylitol can inhibit colonization of certain microorganisms. However, other microorganisms are less susceptible the inhibiting effects of Xylitol. Further, there appears to be a synergistic effect on inhibition when Xylitol and Fluoride are applied simultaneously. S. Palchaudhuri: Raman Spectroscopy of Xylitol Uptake and Metabolism in Gram-Positive and Gram-Negative Bacteria, Applied an Environmental Microbiology, Vol. 77, No. 1, January 2011, pp 131-137.

In my patent, Cloonan, U.S. Pat. No. 6,602,490, incorporated herein by reference, I disclose a dental hygiene rinse including a unique combination in solution of: a sequestrant such as Potassium Citrate; a chelating agent such as Dissodium Edetate; a nonfermentable sweetener such as Xylitol in combination with an emulsifying, wetting and solubizing agent such as Polysorbate, an antioxidant such as Calcium Ascorbate; and, a flavoring agent such as Spearmint and Peppermint. Because the preparation does not contain any effective amount of Sodium Fluoride, Sodium monoflurophosphate, Stanous Fluoride or any other source of Fluoride, it greatly reduces the risk of poisoning. However, in treating mouth wounds the solution provides only a limited antimicrobial effect.

It has been found that the formulation described in the above patent can carry only a limited amount of Xylitol in solution without eventually forming unwanted precipitates which reduce effectiveness and shelf-life. Other negative effects of higher concentration Xylitol include the possible onset of osmotic diarrhoea.

In general, higher concentrations of compounds containing sodium are to be avoided when treating some patients, especially the elderly.

Often extra-oral wound healing is promoted by the use of bandages or other dressings which cover and protect the wound. However, some individuals may have conditions which are exacerbated by the use of bandages. For example, a person may have an allergic reaction to certain adhesives used in bandages, or the skin surrounding a wound may be overly sensitive. Moreover, scabs sometimes form which adhere to the bandage. When the bandage is removed, the wound may be opened afresh.

Some disinfectant and antimicrobial agents irritate sensitive nerve endings near wounds and cause pain. Reducing the pain associated with use of such formulations is desirable.

Therefore, there is a need for improved topical wound healing preparations for use both intra-orally and extra-orally that address the above drawbacks. It would be useful to provide a solution which provides the benefits of higher concentrations of Xylitol without the negative effects described above and to enhance its antimicrobial function. It would be beneficial to maintain a more healthful mouth pH level for patients anticipating and recovering from surgery.

### SUMMARY

The principal and secondary objects of the invention are to provide improved topical treatment of wounds.

These and other valuable objects are achieved by a preparation that combines a higher concentration of Xylitol with another non-Xylitol anti-microbial sweetener.

In some embodiments there is provided an oral and extra-oral topical wound treatment gel which comprises: water; Xylitol in a concentration of between about 1 gram and 3 grams per liter water; Carboxymethylcellulose sodium high viscosity in a concentration of between about 25 grams and 100 grams per liter water; and, an additional antimicrobial, essentially sugarless sweetening agent having a different molecular structure than Xylitol.

In some embodiments, said additional antimicrobial sweetening agent is selected from the group consisting essentially of: DL Menthol, Peppermint, Sorbitol, and Glycerin. In some embodiments, said additional antimicrobial sweetening agent comprises DL Menthol, Peppermint, Sorbitol and Glycerin. In some embodiments, said additional antimicrobial sweetening agent comprises DL Menthol in a concentration of between about 95 grams and 275 grams per liter water. In some embodiments, said additional antimicrobial sweetening agent is selected from the group consisting essentially of: DL Menthol in a concentration of between about 95 milligrams and 275 milligrams per liter water; Peppermint in a proportion of between about 0.4 milliliters and 3.0 milliliters per liter water; Sorbitol in a proportion of between about 2.5 milliliters and 7.5 milliliters per liter water; and, Glycerin in a proportion of between about 95 milliliters to 275 milliliters per liter water. In some embodiments, said additional antimicrobial sweetening agent is selected from the group consisting essentially of: DL Menthol in a concentration of between about 100 milligrams and 200 milligrams per liter water; Peppermint in a proportion of between about 0.4 milliliters and 1.5 milliliters per liter water; Sorbitol in a proportion of between about 2.5 milliliters and 5 milliliters per liter water; and, Glycerin in a proportion of between about 100 milliliters and 200 milliliters per liter water. In some embodiments, said additional antimicrobial sweetening agent comprises: DL Menthol in a concentration of between about 100 milligrams and 200 milligrams per liter water; Peppermint in a proportion of between about 0.4 milliliters and 1.5 milliliters per liter water; Sorbitol in a proportion of between about 2.5 milliliters and 5 milliliters per liter water; and, Glycerin in a proportion of between about 100 milliliters and 200 milliliters per liter water.

In some embodiments, the gel further comprises: a drying/hydrophobic agent comprising Sodium Bicarbonate in a proportion of between about 1 gram to 5 grams per liter water. In some embodiments, the gel further comprises: an astringent selected from the group consisting essentially of: Zinc Acetate in a proportion of between about 100 milligrams to 200 milligrams per liter water; and, Lactoferrin in a proportion of between about 200 milligrams and 750 milligrams per liter water. In some embodiments, the gel further comprises: an additional healing promoter comprising Aloe Vera Extract in a proportion of between about 95 milliliters and 250 milliliters per liter water. In some embodiments, the gel further comprises a preservative comprising Disodium Edetate. In some embodiments, the gel further comprises an emulsifying agent taken from a group consisting essentially of Poloxamer, Polysorbate, and Potassium Citrate. In some embodiments, the gel further comprises an antioxidant taken from a group consisting essentially of Calcium Ascorbate. In some embodiments, the gel further comprises an additional sweetener taken from a group consisting essentially of Spearment, Stevia Extract, Pectin, Aspertame, and Sodium Saccharin Dihydrate. In some embodiments, the gel further comprises a solvent taken from a group consisting essentially of Ethyl Alcohol, Propylene Glycol and Ethylene Glycol. In some embodiments, the gel further comprises a pH-adjusting compound in proportion necessary to set a pH of approximately 7.0.

In some embodiments, the gel is formulated to be ingestable.

In some embodiments, the gel is formulated to form an occlusive dressing and wherein a dried amount of said gel is soluble.

In some embodiments of the gel there is an absence of any effective amount of free fluoride-providing compound.

In some embodiments, there is provided an intra-oral and extra-oral hygiene liquid preparation includes: Water; Xylitol in a proportion of at least 1 gram per liter water; a chelating agent comprising Disodium Edetate in a proportion of between about 3.5 milligram and 8 milligram per liter water; one or more emulsifying, wetting and solubizing agent comprising Polysorbate 20 in a proportion of between about 0.65 milliliter and 2.2 milliliter per liter water, and Poloxamer 338 in a proportion of between about 95 milligrams and 275 milligrams per liter water; and, a non-Xylitol, essentially sugarless, antimicrobial sweetening agent.

In some embodiments, said non-Xylitol antimicrobial sweetening agent is taken from the group consisting essentially of: DL-Menthol in a proportion of between about 95 milligrams and 275 milligrams per liter water; oil of Peppermint in a proportion of between about 0.4 milliliters and 1.5 milliliters per liter water; Sorbitol in a proportion of between about 1.5 milliliters and 2.5 milliliters per liter water; and, Glycerin in a proportion of between about 1.5 milliliters to 5 milliliters per liter water.

In some embodiments, the preparation further comprises: a sequestrant comprising Potassium Citrate in a proportion of between about 95 milligrams and 275 milligrams per liter water; an antioxidant comprising Calcium Ascorbate in a proportion of between about 95 milligrams and 275 milligrams per liter water; an additional sweetener comprising Stevia Extract in a proportion of between about 1 gram and 4.5 grams per liter water; an additional flavoring agent comprising Spearmint (rectified) in a proportion of between about 0.4 milliliter and 1.5 milliliter per liter water; an amount of Ethyl Alcohol in a proportion of between about 3 milliliters and 10 milliliters per liter water; a coloring agent comprising Blue Coloring F.D. & C. #1 in a proportion of between about 0.03 milliliter and 0.06 milliliter per liter water; and, a pH adjusting agent comprising Sodium Carbonate in a proportion of between about 0.001 milliliter and 0.004 milliliter per liter water to adjust the pH of the entire preparation to neutral.

In some embodiments, there is provided a method for avoiding aspiration pneumonia which comprises: administering separate amounts of said preparation to said patient's mouth after each meal during a preoperative period; performing a surgery; and, administering separate amounts of said preparation to said patient's mouth after each meal during a recovery period.

In some embodiments, there is provided a method for treating a topical skin condition which comprises: identifying a first a skin condition in a patient wherein said skin condition is selected from the group consisting of: superficial cuts, abrasions, lacerations, burns, blisters, rashes, acne blemishes, exema patches, psoriasis patches, fungal infections, diaper rash zones, sunburns, dandruff patches, hemorrhoidal tissues, insect bites, insect stings, and poisonous plant contact zones; cleaning a patch skin exhibiting said condition; coating said patch of skin with a continuous layer of said gel; and, allowing said layer to dry in air to form an occlusive seal over said patch.

In some embodiments, the method further comprises: removing said dried gel by washing alone.

In some embodiments, there is provided a method for dental care comprises: rinsing the mouth with a first amount of the preparation including vigorous swishing through the teeth; brushing the teeth, gums and tongue with a dry brush; flossing the teeth in the standard manner; and second rinsing with a second amount of the preparation including vigorous swishing through the teeth.

The content of the original claims is incorporated herein by reference as summarizing features in one or more exemplary embodiments.

### DESCRIPTION OF THE EXEMPLARY EMBODIMENTS

An exemplary embodiment of the wound treatment gel formulation will now be described which includes:

Water;

Xylitol in a proportion of at least 1 gram per liter water, more preferably between about 1 gram to 5 grams per liter water, more preferably between about 1 gram and 3 grams, and most preferably between about 1 gram and 2.5 grams;

Carboxymethyl-cellulose sodium high viscosity as primarily a biocompatible carrier medium providing binding, emulsification, stabilization functions in a concentration of between about 25 grams and 150 grams per liter water, and more preferably between about 30 grams to 100 grams; depending on desired viscosity; and,

A non-Xylitol, essentially sugarless, antimicrobial sweetening agent.

The non-Xylitol, essentially sugarless, antimicrobial sweetening agent can be fulfilled by one or more of the following components:

DL-Menthol as primarily an antimicrobial sweetener, and analgesic and flavoring agent in a concentration of between about 95 milligrams and 275 milligrams per liter water, and more preferably between about 100 milligrams and 200 milligrams per liter water;

Oil of Peppermint as primarily an antimicrobial sweetener, and as an additional flavoring agent in a proportion of between about 0.4 milliliters and 3.0 milliliters per liter water, and more preferably between about 0.4 milliliters and 1.5 milliliters per liter water;

Sorbitol as primarily an antimicrobial sweetener, and as an additional emulsifying agent in a proportion of between about 2.5 milliliters and 7.5 milliliters per liter water, and more preferably between about 2.5 milliliters and 5 milliliters per liter water; and,

Glycerin as primarily an antimicrobial sweetener, and as a humectant in a proportion of between about 95 milliliters to 275 milliliters per liter water, and more preferably between about 100 milliliters and 200 milliliters per liter water.

The wound treatment gel formulation also, preferably, includes:

A drying/hydrophobic agent such as Sodium Bicarbonate in a proportion of between about 1 gram to 6 grams per liter water, and more preferably between about 1 gram and 4 grams per liter water;

An astringent such as Zinc Acetate which can also act as an additional binding in a proportion of between about 100 milligrams to 300 milligrams per liter water, and more preferably between about 100 milligrams and 200 milliliters per liter water; and Lactoferrin in a proportion of between about 200 milligram to 950 milligrams per liter water, and more preferably between about 200 milligrams and 750 milligrams per liter water;

An additional healing promoter agent such as Aloe Vera Extract in a proportion of between about 95 milliliters to 300 milliliters per liter water, and more preferably between about 95 milliliters and 250 milliliters per liter water; and,

A pH adjusting agent such as Sodium Carbonate, Anhydrous which can be added if necessary to adjust the pH to neutral.

It is preferred to use stock Xylitol powder of at least 99% purity. Such Xylitol powder is commercial available from Spectrum Chemicals of Los Angeles, Calif.

The preferred Aloe Vera Extract is derived from aloe vera barbadensis mill and is commercially available from the Silverthorn Ranch Nursery of Fallbrook, Calif.

It shall be noted that Green Tea Extract can be used as an additional healing promoter agent.

In addition to its astringent and binding properties, Zinc Acetate also provides the role of being an antioxidant. Another candidate for the antioxidant role would be Calcium Ascorbate.

A small amount of alcohol, such as Ethyl Alcohol can act as a solvent but also have antimicrobial and known biocompatibility effects. Other possible candidates for the solvent would be Propylene Glycol and Ethylene Glycol.

An additional surfactant detergent and solubulizing agent may be used such as Polysorbate 20, Poloxamer 338, and/or Ethylene Oxide.

An additional preservative such as Disodium Edetate can also be included.

Other gelling and antimicrobial agents include Cellulose Gum Tree extract, or Pectin in a proportion of between about 1 gram to 6 grams per liter water, and more preferably between about 1 gram and 5 grams per liter water.

Natural glycerin is preferably used to act as an additional humectant and to also act as a plasticizer, emolliant and tonicity agent.

Other emulsifying agents can include Potassium Citrate.

One or more of the above functional components can be fulfilled by a single chemical or group thereof.

It is believed that the combination of higher concentration Xylitol with one or more other non-Xylitol, non-sugar-based sweeteners which have their own anti-microbial characteristics provide a synergistic effect at reducing the colonization of many commonly occurring pathogens. Those pathogens which are less susceptible to Xylitol alone, where Xylitol has limited effect, or where the combination of Xylitol and Fluoride has shown some synergistic effectiveness may be better targeted by the additional molecular structures present in the plurality of antimicrobial sweeteners but without the potential negative effects of Fluoride. Combining at least two or even three or more of the above non-Xylitol, non-sugar-based, antimicrobial sweeteners with the high concentration Xylitol may further enhance the ability of the formulation to target myriad pathogens.

Once dried the gel forms an occlusive dressing over the wound. In this way the gel helps prevent the entrance of foreign material including pathogens into the wound. Further, the continued presence of higher concentrations of Xylitol and other antimicrobial sweeteners appear to inhibit colonization of those pathogens which do make it through the dressing.

Another advantage of the gel is it remains substantially soluble in water even after it has dried. In this way the dried gel can be washed off during bathing without peeling and potentially mechanically disturbing the wound.

Another advantage of the above gel formulation is that it can be formulated to be ingestable, and therefore used both orally and extra-orally. Care must be taken to reduce the concentration of possible toxic components for gel formulations which are to be used orally.

For oral use the gel can preferably include an additional sweetening agents such as Stevia Extract from the stevia rebaudiana plant, Pectin, Aspartame and Sodium Saccharin Dihydrate, as well as some additional flavoring agent such as Spearmint.

The composition of a preferred example of the topical wound treatment gel is provided in Table 1.

TABLE 1

| | | |
|---|---|---|
| Water (purified) | 1 | liter |
| Xylitol F.C.C. | 1.3 | gram |
| Carboxymethyl-cellulose sodium | 30 | grams |
| (high viscosity U.S.P.) | | |
| DL-Menthol Crystal U.S.P./N.F. | 100 | milligram |
| Natural Glycerin U.S./N.F. | 100 | milliliter |
| Pectin | 1.5 | gram |
| Sorbitol (D-Glucitol) 70% Solution U.S.P. | 3 | milliliter |
| Zinc Acetate | 140 | milligrams |
| Lactoferrin | 300 | milligrams |
| Aloe Vera Extract | 100 | milliliter |
| Sodium Bicarbonate | 1.5 | gram |

The gel preparation in the above example was obtained according to the following steps:

1) Select an amount of purified water and heat to 125 degrees Fahreheit;
2) Successively stir in Glycerin, DL-Menthol, Aloe Vera, Xylitol, Sodium Bicarbonate, Sorbitol, Pectin, Zinc Acetate, Lactoferrin, and Carboxymethylcellulose;
3) Mix the solution using a powered mixer at (medium speed) for approximately 15 minutes while allowing to cool to room temperature;
4) Cover and refrigerate at approximately 40 degrees Fahrenheit overnight; and,
5) Before packaging for commercial distribution, the preparation is optionally passed through an ultra-violet disinfection unit.

An exemplary embodiment of an improved dental hygiene preparation solution includes: water;

Xylitol in a proportion of at least 1 gram per liter water, more preferably in a proportion of between about 1 gram to 5 grams per liter water, and most preferably between about 1 gram to 4.5 gram per liter of water;

A chelating agent such as a saturated solution of Disodium Edetate (wherein 100 milligrams of Disodium Edetate powder is mixed in 10 milliliter of warm purified water) in a proportion of between about 3.5 milligrams to 8 milligrams per liter water, more preferably in a proportion of between about 3.5 milligrams to 6 milligrams per liter water;

One or more emulsifying, wetting and solubizing agent such as Polysorbate 20 in a proportion of between about 0.65 milliliter and 2.2 milliliter per liter water, and Poloxamer 338 in a proportion of between about 95 milligrams and 275 milligrams per liter water; and,

One or more non-Xylitol, essentially sugarless, antimicrobial sweeteners.

The non-Xylitol, non-sugar-based, antimicrobial sweetener can be fulfilled by one or more of the following components:

DL-Menthol as primarily an antimicrobial sweetener, and analgesic and flavoring agent in a concentration of between about 95 milligrams and 275 milligrams per liter water, and more preferably between about 100 milligrams and 200 milligrams per liter water;

Oil of Peppermint as primarily an antimicrobial sweetener, and as an additional flavoring agent in a proportion of between about 0.4 milliliters and 1.5 milliliters per liter water;

Sorbitol as primarily an antimicrobial sweetener, and as an additional emulsifying agent in a proportion of between about 1.5 milliliters and 2.5 milliliters per liter water; and,

Glycerin as primarily an antimicrobial sweetener, and as a humectant in a proportion of between about 1.5 milliliters to 5 milliliters per liter water.

The preparation solution also, preferably, includes:

A sequestrant such as Potassium Citrate in a proportion of between about 95 milligrams and 275 milligrams per liter water;

An antioxidant, such as Calcium Ascorbate, in a proportion of between about 95 milligrams and 275 milligrams per liter water;

An additional sweetener such as Stevia Extract in a proportion of between about 1 gram and 4.5 grams per liter water;

An additional flavoring agent such as Spearmint (rectified) in a proportion of between about 0.4 milliliter and 1.5 milliliter per liter water;

A small amount of Ethyl Alcohol, as primarily a solvent having antimicrobial and known biocompatibility effects, in a proportion of between about 3 milliliters and 10 milliliters per liter water;

A coloring agent such as Blue Coloring F.D. & C. #1 in a proportion of between about 0.03 milliliter and 0.06 milliliter per liter water; and,

A pH adjusting agent such as Sodium Carbonate in a proportion of between about 0.001 milliliter and 0.004 milliliter per liter water to adjust the pH to neutral.

One or more of the above function components can be fulfilled by a single chemical or group thereof.

The preparation does not contain any effective amount of Fluoride compound that could provide free Fluoride as a caries-preventive agent.

Astaxanin can be used as an additional or replacement preservative.

Other potential candidates for the sequestrant include Sodium Citrate and/or Sodium Tripolyphospate. However, these chemicals would less preferably add sodium to the overall solution.

Other potential candidates for the antioxidant include various green tea extracts.

Other potential candidates for the additional sweetener include Aspartame such as SPLENDA brand sweetener, and/or Sodium Saccharin Dihydrate.

The composition of a preferred example of the dental hygiene liquid preparation is provided in Table 2.

TABLE 2

| | | |
|---|---|---|
| Water (purified) | 1 | liter |
| Xylitol F.C.C. | 1.3 | gram |
| Disodium Edetate U.S.P./N.F. | 4 | milligram |
| Polysorbate 20 U.S.P./N.F. | 0.75 | milliliter |
| Poloxamer 338 U.S.P./N.F. | 100 | milligram |
| Natural F.C.C. Oil of Peppermint | 0.5 | milliliter |
| DL-Menthol Crystal U.S.P./N.F. | 100 | milligram |
| (Crystals are Mixed into the alcohol | | |
| solution). | | |
| Calcium Ascorbate Dihydrate | 100 | milligrams |
| Potassium Citrate F.C.C. (Granules are | 100 | milliliter |
| mixed into water). | | |
| Natural Glycerin U.S./N.F. | 1.5 | milliliter |
| Rectified F.C.C. Oil of Spearmint | 0.5 | milliliter |
| Stevia Extract | 1.3 | gram |
| Ethanol 200 Proof U.S.P./N.F. | 4 | milliliter |
| Blue Coloring F.D. & C. #1 | 0.03 | milliliter |

| | |
|---|---|
| Sodium Carbonate, Anhydrous U.S.P./N.F. | to adjust |
| | pH to 7.0 |

In the example, Xylitol, derived from the cell walls of plants, as been proven to inhibit the growth of bacteria, in particular Strep Mutans, the main bacteria responsible for dental caries. Contrary to Fluoride, Xylitol can be safely ingested. Potassium Citrate is preferred as a sequestrant due to its solubizing and pH-stablilizing properties.

The above formulation provides for the Xylitol content beyond 1 gram per liter water without appreciable precipitation and a shelf life of at least one year.

The preparation in the example was obtained according to the following steps:

Heating the water to approximately 66 degrees Celsius (150° F.);

Dissolving the oil of Spearmint, oil of Peppermint, DL-Menthol and Glycerin in the Ethanol, and stirring the mixture into the heated water. The Polysorbate 20, Poloxamer 338 and Potassium Citrate were then sequentially dissolved into the heated water. Next, the Disodium Edetate was added. After letting the preparation cool to approximately 49 degrees Celsius (120° F.), the Xylitol was added. After letting the preparation cool to approximately 43 degrees Celsius (110° F.), the Calcium Ascorbate, the then the Stevia is added. When the preparation cooled to approximately 38 degrees Celsius (100° F.), the blue coloring agent is added. The pH was then adjusted to 7.0 with Sodium Carbonate.

It should be noted that the preparation was continuously stirred during the entire process. Finally, the preparation was filtered through a 616-20 paper.

Before bottling for commercial distribution, the preparation is passed through an ultra-violet disinfection unit then further filtered through a 5 micron filter prior to bottling.

The wound treatment gel can be conveniently manufactured by replacing the water component in the above embodiment with the above described dental hygiene solution which contains a significant amount of water. In this way, the preferred synergistic mixing of Xylitol and the additional non-Xylitol, antimicrobial sweetener will have already been made.

Therefore, the wound treatment gel can include:

an amount of the above described dental hygiene solution;

Carboxymethyl-cellulose sodium high viscosity as primarily a biocompatible carrier medium providing binding, emulsification, stabilization functions in a concentration of between about 25 grams and 150 grams per liter water, and more preferably between about 30 grams to 100 grams; depending on desired viscosity.

The wound treatment gel formulation manufactured using the dental hygiene solution in place of water also, preferably, includes:

A drying/hydrophobic agent such as Sodium Bicarbonate in a proportion of between about 1 gram to 6 grams per liter water, and more preferably between about 1 gram and 4 grams per liter water;

An astringent such as Zinc Acetate which can also act as an additional binding in a proportion of between about 100 milligrams to 300 milligrams per liter water, and more preferably between about 100 milligrams and 200 milliliters per liter water; and Lactoferrin in a proportion of between about 200 milligram to 950 milligrams per liter water, and more preferably between about 200 milligrams and 750 milligrams per liter water;

An additional healing promoter agent such as Aloe Vera Extract in a proportion of between about 95 milliliters to 300 milliliters per liter water, and more preferably between about 95 milliliters and 250 milliliters per liter water; and,

A pH adjusting agent such as Sodium Carbonate which can be added if necessary to adjust the pH to neutral.

For oral use the gel can preferably include an additional level of sweetener and/or another flavoring agent. Additional Natural glycerin is preferably added as a humectant, plasticizer, emollient and tonicity agent. The binding agent can be augmented with an amount of cellulose gum tree extract, colloid, starch and Sorbitol. Additional sweetening and antimicrobial effect can be provided by additional Sorbitol, and/or Pectin because solubility is less of a problem in the gel than in the liquid solution. A small amount of alcohol and a coloring agent may also be included.

The composition of a preferred example of the topical wound treatment gel is provided in Table 3.

TABLE 3

| | | |
|---|---|---|
| Dental hygiene liquid from Table 2 | 1 | liter |
| Carboxymethyl-cellulose sodium | 30 | grams |
| (high viscosity U.S.P.) | | |
| Natural Glycerin U.S./N.F. | 100 | milliliter |
| Pectin | 1.5 | gram |
| Sorbitol (D-Glucitol) 70% Solution U.S.P. | 3 | milliliter |
| Zinc Acetate | 140 | milligrams |
| Lactoferrin | 300 | milligrams |
| Aloe Vera Extract | 100 | milliliter |
| Sodium Bicarbonate | 1.5 | gram |

Oral Hygiene Maintenance

A preferred oral care regime is described which includes at least once a day cleaning the mouth using a “swish-brush-swish” process.

A cleaning session begins by first rinsing with a first amount of the liquid preparation described in Table 2. The amount is typically between about 5 and 10 milliliters, depending on the size of the individual. Rinsing should continue for a duration of between about 30 seconds, including preferably vigorous swishing through the teeth.

Next, with or without expectorating the preparation, and without using toothpaste, brush the teeth, gums and tongue in the standard fashion using a dry toothbrush.

After brushing expectorate any significant amount preparation remaining in the mouth.

Next, flossing the teeth in the standard manner.

After flossing, perform a second rinsing with a second amount of the preparation described in Table 2, again for a duration of about 30 seconds, and including preferably vigorous swishing through the teeth.

After the second swishing step, expectorate any remaining preparation and do not rinse the mouth.

This completes the cleaning session. After cleaning, wait at least an hour before eating or drinking. There should be no eating or drinking unless followed by another cleaning session before sleep.

The above regime has been found to result in superior oral health.

Extra-Oral Wound Care Regime

A preferred extra-oral wound care regime is described.

First, the type of topical wound should be identified whether it's a superficial cut, abrasion, laceration, burn, blister or rash, or some skin condition resulting from acne, exema, psoriasis, an insect bite or sting, contact with a poisonous plant, hemorrhoids, a fungal infection, diaper rash, sunburn, or dandruff, for example.

Next the patch of skin exhibiting the wound should typically be cleaned, such as though washing with soup and water;

Next the patch is coated with a layer of a gel. The amount should be selected to form a thin, continuous, film to entirely cover over the target area.

Once applied the gel should then be allowed to air dry.

With daily washing the water soluble dried gel will be washed away, and should be reapplied.

The above regime has been found to enhance wound healing times.

Oral pH Management of Surgery Patient Regime

A preferred oral pH management regime for individuals undergoing surgery is described.

Preferably, a patient slated for surgery should receive a dental check up, cleaning and evaluation of oral health to determine whether there is a condition which would discourage the planned surgery. For example, if the patient has an obvious abscess in the mouth or other periodontal condition that needs to be preliminarily treated, the surgery may need rescheduling. For example, if a patient's pocket depth is found to be beyond certain acceptable limits the patient may have pH in the acid range and a correspondingly higher concentration of pathogenic microbes in the sub-gingival tissues. The presence of such a high concentration of pathogenic microbes could increase the chances of postoperative infection.

During a preoperative period of at least 1 week prior to surgery, begin the above described oral hygiene maintenance regime.

Immediately prior to surgery, spray the lips and oral cavity with an atomized amount of the liquid preparation according to Table 2. The spraying can be accomplished by loading the solution into a pump sprayer bottle which delivers between about 0.1 to 0.5 milliliter of solution in an atomized form with each pump.

It should be noted that during surgery the pH level in the mouth will typically drop due to some dehydration of the patient.

Immediately after surgery again apply an atomized amount of the solution to the lips and oral cavity.

If the surgery involves the use of an endotracial tube, particular care should be taken to spray an amount of the solution on those mouth parts such as the hard and soft palate that have been irritated by the tube.

If the patient is recovering from general anesthesia and is unconscious or exhibits some grogginess, the head should be kept elevated to help prevent oral secretions from draining into the subglotal area where they can foster bacterial growth. Further, a suction machine should be used for those patients who have difficulty expectorating.

Once the patient begins eating, then the “swish-brush-swish” oral maintenance regime should be followed during the post-operative period.

Vehicles

The above formulations can be easily adapted to various existing vehicles of delivery which can include, but are not limited to: lotions, ointments, creams, sunburn treating preparations, antifungal treating preparations, lip balm, chewing gum, sanitary wipes, shampoos, suppositories, dental floss, disposable toothbrushes, dental massagers, dental stain removing preparations, liquid jet dental cleaners, mouth sprayers, nasal irrigators, nasal sprayers and nasal inhalers.

For example, solution impregnated floss can be manufactured by soaking a fluid permeable floss material in the solution of Table 2 until the material becomes saturated, then allowing the floss material to dry. Once dry, the floss can be loaded onto the floss dispenser and sold.

In an additional example, a gel impregnated toothbrush can be manufactured by injecting an amount of the gel of Table 1 between the bristles. The impregnated brush can then be packaged and sold.

While the exemplary embodiments of the invention have been described, modifications can be made and other embodiments may be devised without departing from the spirit of the invention and the scope of the appended claims.

What is claimed is:

1. An oral and extra-oral topical wound treatment gel which comprises:
water;
Xylitol in a concentration of between about 1 gram and 3 grams per liter water;
Carboxymethylcellulose sodium high viscosity in a concentration of between about 25 grams and 100 grams per liter water; and,
an additional antimicrobial, essentially sugarless sweetening agent having a different molecular structure than Xylitol.

2. The gel of claim 1, wherein said additional antimicrobial sweetening agent is selected from the group consisting essentially of: DL Menthol, Peppermint, Sorbitol, and Glycerin.

3. The gel of claim 1, wherein said additional antimicrobial sweetening agent comprises DL Menthol, Peppermint, Sorbitol and Glycerin.

4. The gel of claim 1, wherein said additional antimicrobial sweetening agent comprises DL Menthol in a concentration of between about 95 grams and 275 grams per liter water.

5. The gel of claim 1, wherein said additional antimicrobial sweetening agent is selected from the group consisting essentially of:
DL Menthol in a concentration of between about 95 milligrams and 275 milligrams per liter water;
Peppermint in a proportion of between about 0.4 milliliters and 3.0 milliliters per liter water;
Sorbitol in a proportion of between about 2.5 milliliters and 7.5 milliliters per liter water; and,
Glycerin in a proportion of between about 95 milliliters to 275 milliliters per liter water.

6. The gel of claim 1, wherein said additional antimicrobial sweetening agent is selected from the group consisting essentially of:
DL Menthol in a concentration of between about 100 milligrams and 200 milligrams per liter water;
Peppermint in a proportion of between about 0.4 milliliters and 1.5 milliliters per liter water;
Sorbitol in a proportion of between about 2.5 milliliters and 5 milliliters per liter water; and,
Glycerin in a proportion of between about 100 milliliters and 200 milliliters per liter water.

7. The gel of claim 1, wherein said additional antimicrobial sweetening agent comprises:
DL Menthol in a concentration of between about 100 milligrams and 200 milligrams per liter water;
Peppermint in a proportion of between about 0.4 milliliters and 1.5 milliliters per liter water;
Sorbitol in a proportion of between about 2.5 milliliters and 5 milliliters per liter water; and,
Glycerin in a proportion of between about 100 milliliters and 200 milliliters per liter water.

8. The gel of claim 1, which further comprises:
a drying/hydrophobic agent comprising Sodium Bicarbonate in a proportion of between about 1 gram to 5 grams per liter water.

9. The gel of claim 1, which further comprises:
an astringent selected from the group consisting essentially of:
Zinc Acetate in a proportion of between about 100 milligrams to 200 milligrams per liter water; and,
Lactoferrin in a proportion of between about 200 milligrams and 750 milligrams per liter water.

10. The gel of claim 1, which further comprises:
an additional healing promoter comprising Aloe Vera Extract in a proportion of between about 95 milliliters and 250 milliliters per liter water.

11. The gel of claim 1, which further comprises a preservative comprising Disodium Edetate.

12. The gel of claim 1, which further comprises an emulsifying agent taken from a group consisting essentially of Poloxamer, Polysorbate, and Potassium Citrate.

13. The gel of claim 1, which further comprises an antioxidant taken from a group consisting essentially of Calcium Ascorbate.

14. The gel of claim 1, which further comprises an additional sweetener taken from a group consisting essentially of Spearment, Stevia Extract, Pectin, Aspertame, and Sodium Saccharin Dihydrate.

15. The gel of claim 1, which further comprises a solvent taken from a group consisting essentially of Ethyl Alcohol, Propylene Glycol and Ethylene Glycol.

16. The gel of claim 1, which further comprises a pH-adjusting compound in proportion necessary to set a pH of approximately 7.0.

17. The gel of claim 1, which is formulated to be ingestable.

18. The gel of claim 1, which is formulated to form an occlusive dressing and wherein a dried amount of said gel is soluble.

19. The gel of claim 1, in the absence of any effective amount of free fluoride-providing compound.

20. An intra-oral and extra-oral hygiene liquid preparation includes:
Water;
Xylitol in a proportion of at least 1 gram per liter water;
A chelating agent comprising Disodium Edetate in a proportion of between about 3.5 milligram and 8 milligram per liter water;
One or more emulsifying, wetting and solubizing agent comprising Polysorbate 20 in a proportion of between about 0.65 milliliter and 2.2 milliliter per liter water, and Poloxamer 338 in a proportion of between about 95 milligrams and 275 milligrams per liter water; and,
A non-Xylitol, essentially sugarless, antimicrobial sweetening agent.

21. The preparation of claim 20, wherein said non-Xylitol antimicrobial sweetening agent is taken from the group consisting essentially of:
DL-Menthol in a proportion of between about 95 milligrams and 275 milligrams per liter water;
Oil of Peppermint in a proportion of between about 0.4 milliliters and 1.5 milliliters per liter water;
Sorbitol in a proportion of between about 1.5 milliliters and 2.5 milliliters per liter water; and,
Glycerin in a proportion of between about 1.5 milliliters to 5 milliliters per liter water.

22. The preparation of claim 20, which further comprises:
a sequestrant comprising Potassium Citrate in a proportion of between about 95 milligrams and 275 milligrams per liter water;
an antioxidant comprising Calcium Ascorbate in a proportion of between about 95 milligrams and 275 milligrams per liter water;
an additional sweetener comprising Stevia Extract in a proportion of between about 1 gram and 4.5 grams per liter water;
an additional flavoring agent comprising Spearmint (rectified) in a proportion of between about 0.4 milliliter and 1.5 milliliter per liter water;
an amount of Ethyl Alcohol in a proportion of between about 3 milliliters and 10 milliliters per liter water;
a coloring agent comprising Blue Coloring F.D. & C. #1 in a proportion of between about 0.03 milliliter and 0.06 milliliter per liter water; and,
a pH adjusting agent comprising Sodium Carbonate in a proportion of between about 0.001 milliliter and 0.004 milliliter per liter water to adjust the pH of the entire preparation to neutral.

23. Using an amount of the liquid preparation of claim 20, a method for avoiding aspiration pneumonia which comprises:
administering separate amounts of said preparation to said patient's mouth after each meal during a preoperative period;
performing a surgery; and,
administering separate amounts of said preparation to said patient's mouth after each meal during a recovery period.

24. Using an amount of the gel of claim 1, a method for treating a topical skin condition which comprises:
identifying a first a skin condition in a patient wherein said skin condition is selected from the group consisting of: superficial cuts, abrasions, lacerations, burns, blisters, rashes, acne blemishes, exema patches, psoriasis patches, fungal infections, diaper rash zones, sunburns, dandruff patches, hemorrhoidal tissues, insect bites, insect stings, and poisonous plant contact zones;
cleaning a patch skin exhibiting said condition;
coating said patch of skin with a continuous layer of said gel; and,
allowing said layer to dry in air to form an occlusive seal over said patch.

25. The method of claim 24, which further comprises:
removing said dried gel by washing alone.

26. Using an amount of the liquid preparation of claim 20, a method for dental care comprises:
rinsing the mouth with a first amount of the preparation including vigorous swishing through the teeth;
brushing the teeth, gums and tongue with a dry brush;
flossing the teeth in the standard manner; and
second rinsing with a second amount of the preparation including vigorous swishing through the teeth.
